(19) United States
(12) Patent Application Publication
(10) Pub. No.: US 2025/0231183 A1
(43) Pub. Date: Jul. 17, 2025

(54) DEVICE AND METHOD FOR ISOLATION AND DETECTION OF TARGETS IN A SAMPLE

(71) Applicant: Massachusetts Institute of Technology, Cambridge, MA (US)

(72) Inventors: Rohit N. Karnik, Cambridge, MA (US); Emily Barret Hanhauser, Brookline, MA (US); Michael Bono, Clifton Park, NY (US); Mary Claire Strawser, Cambridge, MA (US)

(73) Assignee: Massachusetts Institute of Technology, Cambridge, MA (US)

(21) Appl. No.: 18/855,729

(22) PCT Filed: Apr. 11, 2023
(86) PCT No.: PCT/US2023/018149

§ 371 Date: Oct. 10, 2024

Related U.S. Application Data
(60) Provisional application No. 63/329,961, filed on Apr. 12, 2022.

(51) Int. Cl. G01N 33/543; B01L 3/00; G01N 33/569

(52) U.S. Cl. CPC G01N 33/54326; B01L 3/5021; G01N 33/56911; G01N 33/56961; G01N 33/56972; B01L 2200/026; B01L 2200/0668

(57) ABSTRACT

Embodiments disclosed herein relate to methods and devices for detecting targets in a sample. A plurality of targets in a sample and a plurality of particles may be provided in a volume and configured to bind to one another to form bound particle-target complexes. In some embodiments, the volume may also include one or more density gradient or two or more density media which may form an interface. The bound particle-target complexes may experience a density shift and separation in the density media relative to unbound targets and/or unbound particles. In some such embodiments, the bound particle-target complexes may settle at the interface formed by the density media. In some embodiments, the separated bound particle-target complexes may then undergo concentration, extraction, and analysis steps.

### RELATED APPLICATIONS

This application claims the benefit of priority under 35 U.S.C. § 119 (e) of U.S. Provisional Application Ser. No. 63/329,961, filed Apr. 12, 2022, the disclosure of which is incorporated herein by reference in its entirety.

### FIELD

Disclosed embodiments are related to methods and devices for detecting targets in a sample.

### BACKGROUND

Methods and devices for detecting targets in samples have been employed in a variety of applications to provide effective and timely analysis of samples. In particular, using particulate target detection methods for samples may provide timely diagnosis of various diseases, infections, or the like. While such detection methods may be used in a variety of suitable applications, an application of particular importance includes using target detection methods to diagnose patients with sepsis, which is a bloodstream infection that causes organ dysfunction which may prove fatal for a patient if not treated properly and timely. This infection can be caused by bacteria, viruses, fungi, and protozoa, but the most common causative agents are bacteria and viruses. Sepsis may manifest in a variety of symptoms that include pro-inflammatory and anti-inflammatory responses, changes in coagulation of a patient's blood, and alterations in cardiovascular, neuronal, autonomic, hormonal, bioenergetic, and/or metabolic pathways. A diagnosis of sepsis requires identification of an infection and confirming immune dysfunction. The immune dysfunction caused by sepsis is clinically identified by using the Sequential Organ Failure Assessment (SOFA) or quickSOFA. Sepsis is known to be a global health threat, causing millions of deaths worldwide and costing billions of dollars annually. Sepsis is also known to be a particularly complex condition requiring intensive care, and early diagnosis of the pathogen is considered vital to be able to provide early treatment to the diagnosed patient. However, many current diagnostic methods can take several days to yield positive results and may yield inaccuracies that reduce confidence in the diagnosis.

### SUMMARY

In some embodiments, a method of detecting targets in a sample is provided. The method may include binding at least a portion of a plurality of particles to at least a portion of a plurality of targets within a volume such that a plurality of bound particle-target complexes are formed. The density of the bound particle-target complexes may be different than both the unbound targets and the unbound particles. The method may further include separating the bound particle-target complexes from both the unbound targets and the unbound particles using at least one density media.

In some embodiments, a device for detecting targets in a sample is provided. The device may include a volume and at least one density media reservoir configured to contain at least one density media, where the at least one density media reservoir may be in fluid communication with the volume. The device may also include a sample reservoir and the sample reservoir may be configured to contain a sample including a plurality of targets, where the sample reservoir may be in fluid communication with the volume. A plurality of particles may also be disposed in the volume and may be configured to bind to the plurality of targets to form bound particle-target complexes, where the density of the bound particle-target complexes is different than the density of both the unbound targets and the unbound particles.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 presents one embodiment of a device used for loading of density media and a sample comprising targets, according to some embodiments;

FIG. 2A presents a step of adding a particle solution to a sample tube, according to some embodiments;

FIG. 2B presents a step of adding a blood sample to the sample tube of FIG. 2A, according to some embodiments;

FIG. 2C presents a step of allowing the particles and blood sample to incubate in the sample tube of FIG. 2B such that at least a portion of targets in the blood sample bind to at least a portion of the particles, according to some embodiments;

FIG. 3A presents a step of adding the media of FIG. 2C into a device containing two or more density media forming an interface, according to some embodiments;

FIG. 3B presents a step of centrifuging the media of FIG. 3A such that bound particle-target complexes are caught at the interface, according to some embodiments;

FIG. 3C presents a step of magnetically concentrating the bound particle-target complexes of FIG. 3B at an outlet of the device, according to some embodiments;

FIG. 3D presents an illustration of the centrifugal and magnetic forces that may be applied in FIGS. 3B and 3C, respectively, according to some embodiments;

FIG. 4A presents an image of a container including two density media disposed therein, a sample, and a pre-positioned outlet, according to some embodiments;

FIG. 4B presents an enlarged schematic view of the region 4B of FIG. 4A following an incubation step, according to some embodiments;

FIG. 4C presents the embodiment of FIG. 4B following a separation step, according to some embodiments;

FIG. 4D presents an enlarged schematic view of the solution located around the density media interface indicated approximately by region 4D of FIG. 4C following extraction of the solution to a different container followed by a concentration step, according to some embodiments;

FIG. 5A presents an image of a tube showing isolation of bound bacteria in a middle interface, isolation of unbound particles at the bottom of the tube, and isolation of blood cells at the top of the tube, according to some embodiments;

FIG. 5B presents experimental data of FIG. 5A for bacterial count at the top of the tube, at the middle interface, and at the bottom of the tube, according to some embodiments;

FIG. 6 presents experimental data for bacterial count in a buffer with varying bead to bacteria ratios, according to some embodiments;

FIG. 7 presents experimental data for separation of targets using buffer and whole blood, according to some embodiments;

FIG. 8 presents experimental data for flow cytometry of beads and bacteria, according to some embodiments;

FIG. 9 presents an exemplary magnet configuration for a magnetic concentration step; and

### DETAILED DESCRIPTION

Detection of particulate targets in samples such as blood, water, food, or soil is an important public health or environment concern. Target detection methods may be used to provide timely diagnosis and analysis of various diseases, infections, or other suitable conditions. As disclosed herein, an important condition that requires timely and accurate diagnosis is sepsis, which has a high mortality rate and the necessary treatments are more effective if provided at the early onset of infection. In current practice, blood culture is used in suspected cases of sepsis to identify an infection. If a positive case of sepsis is detected, it may often carry a greater mortality risk than certain other infections. In particular, sepsis involves an immune response to infection that causes life-threatening organ dysfunction, and as such, a sepsis diagnosis must be treated timely to help save the life of a patient.

Due to the difficulties in diagnosing sepsis, numbers are likely underreported in developing countries, but it is estimated that between six and nineteen million people are killed every year by sepsis. In the United States alone, sepsis afflicts close to a million people, killing more than 220,000 people each year, and the financial cost approaches 20 billion dollars. The broad-spectrum antibiotics used to treat suspected sepsis patients before a blood culture comes back positive also present a risk of developing drug-resistant micro-organisms and patients who survive sepsis often suffer long-term health effects. The mortality rate of sepsis is estimated to be between 25-50% in the US, and mortality risk becomes elevated by 8% for each hour that a patient waits to get antibiotic administration.

In the current treatment paradigm, patients with suspected sepsis are immediately placed on broad-spectrum antibiotics while a blood culture is being performed. Blood culture is a procedure where a sample of blood is placed in growth media and incubated until a pH or pressure change is detected due to the growth of bacteria. It takes a median time of 15 hours to determine a positive blood culture. For an adult patient, it is recommended to take three blood draws with 20 ml of blood from each draw to have the best chance of a true positive result. For an infant, child, or senior, these kinds of blood volumes are prohibitive. An additional complication to the diagnostic value of blood culture is that patients with previous antibiotic treatment or certain hard-to-grow bacteria may have negative culture results despite the presence of bacteria in the bloodstream. Accordingly, while blood culture is considered to be the standard of detection, the use of blood culture does have flaws.

The inventors have recognized that detecting particulate targets in samples at low concentrations is difficult as the background of the sample is often orders of magnitude more common than the target and distinguishing between targets and the remainder of the sample can only be done if some sort of differentiation can be performed on the targets. Blood culture addresses this problem by facilitating the multiplication of the targets (e.g., target cells). Moreover, while magnetic isolation has been independently deployed to isolate targets, magnetic isolation alone may not sufficiently isolate targets that are of low concentrations in complex media, especially since the targets are typically not separated from the large number of magnetic particles that are typically used for the isolation.

In view of the above, the inventors have recognized that rapid, accurate, and affordable testing and diagnostic methods are needed to allow for isolation and analysis of particulate targets in samples. In particular, isolation of target cells or target particles from a complex media (e.g., blood) into a sufficiently small field of view may be desirable for a number of applications.

In some embodiments, targets may be isolated in a sample by separation and concentration of the targets relative to the background of a complex media. In some such embodiments, the sample includes a plurality of targets. As disclosed herein, a suitable sample may take the form of a variety of complex media such as blood, foodstuffs, soil, water, or any other suitable media. In some embodiments, a plurality of particles may be provided and mixed with the sample including the plurality of targets. The particles may be configured to bind with the targets included in the sample to form bound particle-target complexes. The plurality of particles may be dispersed within a mixture which may be added into the volume where the sample is disposed. The sample comprising the targets and the particles may be introduced into the volume in any suitable order as the disclosure is not so limited. Upon formation of the bound particle-target complexes, the effective density of the targets may by altered. The effective density refers to the mass of the complex divided by the volume of the complex. The density of the bound particle-target complexes may be greater than or less than the density of the unbound targets and/or particles, and this difference in density of the complexed targets may be used to facilitate density separation of the bound complexes relative to the unbound targets and/or the unbound particles.

In some embodiments, the above noted sample including complexed targets and particles may be added to a volume including one or more density media. For example, two density media having different densities may be provided in the volume such that an interface is formed between the two density media. Upon binding of the particles and targets to form bound particle-target complexes, the density of the bound complexes may differ relative to the density of the two density media such that density separation of the bound complexes occurs and the bound complexes settle at a desired location within the volume. In embodiments where two density media are provided, the bound complexes may have a density greater than the first density media and a density less than the second density media such that the bound complexes settle at the interface formed by the two density media due to density separation. While an example including two density media is disclosed above, any suitable number of density media may be used to differentiate bound particle-target complexes in a sample from the background complex media. For example, one, two, three, or any appropriate number of density media each having a different density may be provided within a volume to form one or more interfaces that may be used to isolate the bound target particle complexes from the sample as the disclosure is not limited in this fashion.

As noted above, bound particle-target complexes may have a different density relative to the density media provided within a volume. In some embodiments, only a portion of the particles may bind to a portion of the targets in a sample to form bound particle-target complexes. As such, there may be unbound targets and/or unbound particles remaining within the volume. In some embodiments, the bound particle-target complexes may have a different density relative to the unbound targets and/or the unbound particles such that density separation will occur between the bound complexes and the unbound particles and targets. For example, the density of the bound particle-target complexes may be greater than the density of the unbound targets but less than the density of the unbound particles. In another example, the density of the bound particle-target complexes may be greater than the density of the unbound particles but less than the density of the unbound targets. In these examples, one or more density media may be provided within the volume with densities between the densities of these different components to separate bound complexes from the unbound targets and particles within different portions of the volume. For example, two density media may be provided and form an interface as noted above, and the bound complexes may settle at the interface while the unbound targets and/or unbound particles may settle at the top and/or bottom of the volume depending on the relative densities of these different components.

In some embodiments, more than two density media (e.g., three or more density media with different densities) may be used and multiple populations of particles configured to bind to different targets may be used. In such an embodiment, the densities of the density media and the separate populations of particle-target complexes may be selected to isolate the separate populations of particle-target complexes at separate interfaces between the different gradient density media. Thus, it should be understood that the current disclosure is not limited to any particular number of gradient density media, populations of particles, and/or populations of targets.

While the bound particle-target complexes have been noted above as being separable via density separation, use of a body force may also be provided to assist in separation of the bound particle-target complexes. In some embodiments, a suitable body force may include centrifugal or magnetic forces. In embodiments where centrifugal forces are used, the volume containing the sample may rotate at appropriate speeds to generate sufficient centripetal force to permit separation of the bound particle-target complexes from the unbound targets and the unbound particles. In embodiments where magnetic forces are used, the bound particle-target complexes may be subjected to a magnetic field of sufficient strength which separates the bound complexes from the unbound targets and/or the unbound particles.

While some embodiments disclosed herein describe a method of detecting targets in a sample, a device may also be provided to facilitate isolation and detection of targets in a sample. In some embodiments, the device may include a volume which may be configured to receive at least one density media, a sample comprising a plurality of targets, and/or a plurality of particles. The at least one density media may be initially contained within a density media reservoir which is in fluid communication with the volume. In embodiments where a plurality of density media are used, each density media may be initially contained within a separate density media reservoir. The sample comprising the plurality of targets may be contained within a sample reservoir that is also in fluid communication with the volume. Without wishing to be bound by theory, the density media reservoirs and the sample reservoirs may be in fluid communication with the volume using any suitable fluidic connection, including but not limited to valves such as siphon valves. In some embodiments, the siphon valves may include serpentine channels configured to facilitate delivery of the respective reservoir contents to the volume.

In some embodiments, the inventors have recognized benefits with sequentially loading the density media and the sample to form layers of media within the volume. In some such embodiments, a serpentine channel siphon valve may be employed for each reservoir, and the number of curves may be selectively chosen to increase or decrease the load time of the reservoir contents into the volume, see for example the valve design in “Siegrist, J., Gorkin, R., Clime, L. et al. Serial siphon valving for centrifugal microfluidic platforms. Microfluid Nanofluid 9, 55-63 (2010).” which is incorporated herein in its entirety, though other appropriate valve and/or channel designs may also be used as the disclosure is not so limited. For example, the fluidic connections which include more curves and overall flow path length may take longer for the respective reservoir contents to load into the volume. As such, in some embodiments, the inventors have recognized benefits to tailoring the device such that a bottom density media is loaded first, followed by a top density media, and lastly followed by a sample, all of which may be sequentially loaded into the volume in the above noted sequence. While the above examples are disclosed, the sample and the at least one density media may be simultaneously, sequentially, or otherwise loaded into the volume as the disclosure is not so limited.

In addition to loading one or more density media and a sample comprising targets into the volume, a plurality of particles may also be disposed within the volume. The particles may be provided in the volume prior to loading of the density media and/or sample or following loading of the density media and/or sample as the disclosure is not so limited. According to embodiments disclosed herein, the plurality of particles may be configured to bind to the plurality of targets in the sample to form bound particle-target complexes which have a different density than unbound targets and unbound particles. The bound particle-target complexes may then be separated from the unbound targets and the unbound particles due to a density shift relative to the density media and/or due to the application of a body force (e.g., centrifugal forces or magnetic forces). Following separation of the bound particle-target complexes, the bound complexes may be concentrated, optionally extracted, and analyzed to determine characteristics of the targets in the sample, as is discussed in greater detail below. It should be further understood that any of the embodiments disclosed herein could be embodied as a method or device as the disclosure is not so limited.

The volume containing the at least one density media, the sample, and/or particles may be of any suitable size and/or shape as the disclosure is not so limited. In some embodiments, the volume may be of a cross-sectional shape including, but not limited to circular, ellipsoidal, square, rectangular, or any other suitable shape. In some embodiments, a suitable cross-sectional dimension (e.g., a diameter, width, or other transverse dimension perpendicular to a longitudinal axis or other axis of a container) of the volume may be greater than or equal to 100 nm, 1 μm, 10 μm, 100 μm, 1 mm, 2 mm, 5 cm, 10 cm, 50 cm, or greater. Correspondingly, the cross-sectional dimension may be less than or equal to 100 cm, 50 cm, 10 cm, 5 cm, or other appropriate dimensions. Combinations of the foregoing are contemplated including, for example, a cross-sectional dimension between or equal to 100 nm and 100 cm, or more preferably between 100 nm and 5 cm in some embodiments. Of course dimensions both greater and less than those noted above are also contemplated.

Upon separating and isolating the bound particle-target complexes from the rest of the sample, the bound complexes may optionally be concentrated at a desired location of the volume. Concentration of the bound complexes may be achieved through use of magnetophoresis to facilitate movement of the bound complexes to a desired location under a resulting magnetic field. In some embodiments, the inventors have recognized benefits associated with concentrating the bound complexes at one or more viewing windows, extraction points, or other appropriate locations which may be provided in the volume. For example, one or more viewing windows may be provided on the side of the volume, and the bound complexes may be concentrated at the one or more viewing windows for analysis. In another example, the one or more viewing windows may be provided at the top and/or bottom of the volume. While these examples are disclosed, one or more viewing windows may be provided in any suitable configuration along the volume to view or detect the complexes with or without magnetic concentration, including by imaging, light scattering, or by other suitable methods. In some embodiments, the captured targets may be imaged with single resolution.

In some embodiments, the volume may include an outlet to permit extraction of the isolated bound particle-target complexes. In some such embodiments, magnetophoresis may be used to magnetically concentrate the bound complexes at the outlet. The outlet may be pre-positioned in the volume to correspond to an expected location of an interface at which the complexes may be located. This position may be determined based on the predetermined volumes of materials (e.g., the density media) to be added to a container. In some embodiments, the outlet may be a tube of sufficient size to permit extraction of the bound complexes. In some embodiments, the inventors have recognized particular benefit with positioning the outlet at an interface formed by two or more density media. Such a configuration would allow the bound complexes to settle at the interface and then be easily extracted using suction, and optional concentration at the opening to the outlet. In some embodiments, the bound complexes may be extracted via the outlet to an external reservoir. While one outlet is referenced above, a plurality of outlets may be employed as the disclosure is not so limited.

In some embodiments, a sufficient volume of solution may be extracted from the volume to permit external analysis of the targets in a sample. In some embodiments, a suitable extraction volume may be greater than or equal to 1 nL, 10 nL, 100 nL, 1 μL, 10 μL, 100 μL, 1 mL, 10 mL, 100 mL, or greater. The extraction volume may also be less than or equal to 1 L, 100 mL, 10 mL, 1 mL, or other appropriate dimensions. Combinations of the foregoing are contemplated including, for example, an extraction volume between or equal to 1 nL and 1 L, or more preferably between 1 nL and 1 mL in some embodiments.

To allow for such extraction volumes to be removed for analysis, the outlet may also be of a suitable transverse dimension (e.g., diameter). For example, the outlet may be of a transverse dimension greater than or equal to 100 nm, 1 μm, 10 μm, 100 μm, 1 mm, 5 mm, 1 cm, 2 cm, 5 cm, 10 cm, 50 cm, or greater. The outlet may also be of a transverse dimension less than or equal to 100 cm, 50 cm, 10 cm, 5 cm, or lesser. Combinations of the foregoing are contemplated including, for example, an outlet transverse dimension of between or equal to 100 nm and 100 cm, or more preferably between 100 nm and 5 cm in some embodiments.

In some embodiments, the bound particle-target complexes may be concentrated into a sufficiently small viewing region to permit detection and analysis of the targets in a sample. In some embodiments, the viewing region may be a fluid layer at the bottom of an external container or reservoir, or a well plate where the solution comprising the bound particle-target complexes is deposited. In other embodiments, the bound particle-target complexes may be concentrated into a channel, a well, or any other suitable geometric feature. The viewing regions may have a volume greater than or equal to 1 fL, 10 fL, 100 fL, 1 μL, 10 pL, 100 μL, 1 nL, 10 nL, 100 nL, 1 μL, 10 μL, 100 μL, 1 mL, or greater. The viewing regions may also have a volume less than or equal to 10 mL, 1 mL, 100 μL, 10 μL, or lesser. Combinations of the foregoing are contemplated including, for example, a viewing region volume of between or equal to 1 fL and 10 mL, or more preferably between 1 fL and 10 μL in some embodiments.

Upon extraction, the bound complexes may be detected and analyzed using a variety of external equipment to detect characteristics regarding the targets in the sample. Any appropriate type of analysis using physical, chemical, or biological tests could be used. For example, the presence or absence of targets could be measured by optical, electrical, or magnetic methods. In some embodiments, identification of the targets (e.g., bacteria) present could be achieved using cell culture, biochemical tests or an independent molecular analysis system. The proposed primary embodiment of the method disclosed herein would incorporate a disposable lab-on-a-disk microfluidic module capable of reagent storage, loading of the discrete density media interface, loading of the sample with magnetic beads, centrifugation of the sample, magnetophoretic concentration of the target cells, and optical analysis. The sample loading and magnetic setup positioning would be accomplished in a benchtop device. The lab-on-a-disk would include approximately four of the same devices to detect different populations of target cells. Alternately, detection may also be performed within the volume before extraction. In some embodiments, the isolated targets are imaged using microscopic methods including brightfield, differential interference contrast, fluorescence microscopy, Raman microscopy, scanning electron microscopy, transmission electron microscopy, or other methods at single-cell resolution. In some embodiments, the behavior of the isolated target cells, such as locomotion, cell division, or motility, is imaged at single cell resolution. In other embodiments, isolated targets are analyzed by methods such as atomic force microscopy or mass spectroscopy including MALDI-TOF mass spectroscopy.

Because the target cells/particles are preserved using the techniques disclosed above, the opportunity exists for a wide variety of downstream analyses. Essentially, any current or future analysis technique for single cells can be used. Additionally, the target cells could be cultured for even more diverse analyses. Molecular analyses may also be easily accomplished with the target cell population extracted using the embodiments disclosed herein. In comparison to traditional blood culture or destructive molecular testing, the methods disclosed herein provide more opportunities for querying the targets of interest.

In some embodiments, the inventors have recognized particular benefit in employing the embodiments of the invention disclosed herein to not only detect the presence or absence of bacteria, but also to provide pathogen identification. The proposed disclosure may provide a highly concentrated sample bound to the particles, so several paths to pathogen identification are available. Identification could be performed optically, using Raman spectroscopy or surface enhanced Raman spectroscopy, polymerase chain reaction (PCR) system, immunolabeling, or through classification using biochemical tests and stains, among other forms of analysis as discussed in greater detail below. While the proposed embodiments disclosed herein may be used complementary to blood culture, it may be possible for the proposed invention to replace the slow, unreliable blood culture paradigm.

In some embodiments, any suitable sample may be used including, but not limited to blood, other bodily fluids including urine, cerebrospinal fluid, saliva, etc., water, foodstuffs, soil, water samples from a variety of municipal or environmental sources, or any suitable complex media for which differentiation of targets contained with the complex media may be needed.

The samples may be of any suitable volume as the disclosure is not so limited. In some embodiments, a suitable sample volume to be used may be greater than or equal to 1 μL, 10 μL, 100 μL, 1 mL, 5 mL, 10 mL, 20 mL, 30 mL, 50 mL, 100 mL, or greater. The sample may also be of a volume less than or equal to 1 L, 100 mL, 50 mL, 30 mL, or lesser. Combinations of the foregoing are contemplated including, for example, a sample volume between or equal 1 μL and 1 L, or more preferably between 1 mL and 30 mL in some embodiments. The inventors have recognized that this preferred range may be desirable for certain applications involving sepsis detection and treatment.

In some embodiments, any suitable targets in a sample may be separated and concentrated for analysis including, but not limited to cells, viruses, bacteria, plankton, algae, spores, cysts, pollen, food pathogens, fungi, protozoa, exosomes, cell organelles, microplastics, waste particulates, metal particles, soil particulates, minerals, or any other suitable targets. In some such embodiments, the target cells may include blood cells, cancer cells, immune cells, plant cells or any other suitable cell type.

In some embodiments, any suitable particles may be used to bind to the targets for differentiation including, but not limited to air bubbles, liquid droplets, magnetic particles, core shell particles, metal particles, ceramic particles, plasmonic coated particles, multiphase particles, anti-fouling coated particles, polymer brush coated particles, gels, hydrogels, vesicles, polymersomes, or any other suitable particle type as the disclosure is not so limited. The particles may also have certain properties that allow for differentiation of the targets from the background of the sample once the particles have bound to the targets. In the preferred embodiment disclosed herein, differentiation occurs due to the particles altering the effective density of the targets when formed as a bound particle-target complex such that the bound complexes experience an effective density shift. However, the differentiation of the targets may also be accomplished using charges, surface charges, zeta potential, magnetic properties, dielectric constants, size, shape, or flow properties as the disclosure is not so limited.

In some embodiments, the particles may include microspheres or beads that are used to bind to corresponding targets in a sample. In some embodiments, the targets may be molecular targets. For example, for smaller targets (e.g., toxins, biomarkers, vitamins, nucleic acids, or other molecules) a sandwich assay could be created using two types of beads. In this approach, one of the beads is separated out from the sample and serves as a proxy target. The bead density and size is chosen analogous to the density of the target in the theoretical design considerations described later. To perform this assay, two types of beads are modified with affinity molecules such as antibodies that will cause the beads to adhere to each other in the presence of the targets (molecule, cell, virus, particle, etc.). The beads are incubated with the sample, and the separation process is performed. Only bound beads are isolated, enabling rapid and sensitive detection of the target analyte through detection of bead pairs or clusters. This approach is also applicable to larger (particulate) targets, although the density of the target will need to be accounted for in the theoretical framework described later. For example, the volume of one of the beads and the target could be added and their density averaged to enable their treatment as a larger bead with an appropriate density. In some embodiments, the beads may be functionalized with nucleic acids to detect certain DNA or RNA sequences in the sample, including single nucleotide polymorphisms.

The particles may also be of any suitable density as the disclosure is not so limited. In some embodiments, the density of the particles may be selectively chosen to provide a sufficient change in the overall density of the bound particle-target complexes to facilitate separation of the bound complexes relative to the unbound targets, the unbound particles, and/or the one or more density media. For example, the particles may be of a sufficient density such that the bound particle-target complexes are urged to settle at the interface formed by two density media in a volume, while the unbound targets and unbound particles settle at the top and bottom of the volume, respectively. In another example in which a single density media is used, the unbound targets and unbound particles may instead settle at the bottom and top of the volume, respectively.

The particles, which may be nanoparticles, microparticles, or macroparticles, may be of any suitable size as the disclosure is not so limited. In some embodiments, a size of the particles may be selectively chosen to facilitate binding to the corresponding targets provided in a sample, thereby resulting in a size of the particle varying depending on the size of the corresponding targets. In some embodiments, an average maximum transverse dimension or size (e.g., a diameter or length) of the particles may be greater than or equal to 5 nanometers (nm), 10 nm, 20 nm, 30 nm, 50 nm, 100 nm, 500 nm, 1 micrometers (μm), 5 μm, 10 μm, 50 μm, 100 μm, 200 μm, 300 μm, 500 μm, 750 μm, or greater. In some embodiments, an average maximum transverse dimension of the particles may be less than or equal to 1,000 μm, 750 μm, 500 μm, 300 microns, 200 μm, 100 μm, 50 μm, 10 μm, 5 μm, 1 μm, 500 nm, or lesser. Combinations of the above are contemplated including, for example, particles with average maximum transverse dimensions between or equal to 5 nm and 1,000 μm. The particles provided in a mixture within a sample volume may also not all be of the same size. For example, a portion of the particles may be approximately 500 μm in size while another portion of the particles may be approximately 200 μm in size. The size of the particles may also depend on the size of the targets as noted above. For example, in embodiments where the targets are viruses, the particles may be sized on the order of about 20 nm whereas in embodiments where the targets are plant cells, the particles may be sized on the order of 300 microns. While these above examples are disclosed, any suitable particle size may be employed for use in binding with any suitable targets as the disclosure is not so limited.

The particles may also be of any suitable number for a given application to permit sufficient formation of bound particle-target complexes as the disclosure is not so limited. In some embodiments, a suitable number of particles may be greater than or equal to 100, 103, 104, 105, 106, 107, 108, 1010, 1012, or greater. A suitable number of particles may also be less than or equal to 1014, 1012, 1010, 108, or lesser. Combinations of the foregoing are contemplated including, for example, a number of particles between or equal to 100 and 1014, or more preferably 100 and 108 in some embodiments.

As used herein any reference to particles should be understood to also refer to the potential use of nanoparticles, microparticles, or macroparticles as well as the disclosure is not limited in this fashion. Thus a reference to particles, nanoparticles, microparticles, or macroparticles in the various embodiments disclosed herein should be understood to include the use of only microparticles, only nanoparticles, only macroparticles, both microparticles and nanoparticles, combinations of microparticles and/or nanoparticles with other sized particles and/or any other appropriate combination.

In some embodiments, a number of the particles may be bound to a number of the targets in a sample to create a plurality of bound particle-target complexes. In some such embodiments, the particles may be bound to the targets using any suitable method including, but not limited to electrostatic binding, magnetic binding, capillary force binding, surface adhesion binding, chemical binding, aptamer binding, or any other suitable binding type as the disclosure is not so limited.

The density of the bound particle-target complexes may be determined by determining the volume of a given target (e.g., bacteria) and determining how many particles can be bound to the target. As such, the size, shape, density, or type of particles may be selectively chosen to achieve a desired density for the bound complexes. In some embodiments, a suitable density of the bound complexes may be greater than or equal to 0.5 g/ml, 0.6 g/ml, 0.7 g/ml, 0.8 g/ml, 0.9 g/ml, 0.95 g/ml, 1 g/ml, 1.05 g/ml, 1.1 g/ml, 1.2 g/ml, 1.3 g/ml, 1.4 g/ml, 1.5 g/ml, 1.6 g/ml, 1.7 g/ml, 1.8 g/ml, 1.9 g/ml, 2 g/ml, 2.5 g/ml, 3 g/ml, or greater. In some embodiments, a suitable density of the bound complexes may be less than or equal to 3.1 g/ml, 3 g/ml, 2.5 g/ml, 2 g/ml, or lesser. Combinations of the foregoing are contemplated including, for example, a bound complex density between or equal to 0.5 g/ml and 3.1 g/ml, or more preferably between 0.5 g/ml and 2 g/ml in some embodiments.

As disclosed herein, during the formation of bound particle-target complexes, unbound targets and/or particles may remain in the volume. In some embodiments, a fraction of the total targets in a volume that are bound to particles may be greater than or equal to 0.1%, 1%, 5%, 10%, 20%, 50%, 80%, 90%, 95%, 99%, or other appropriate percentage. Accordingly, in some embodiments, the fraction of targets from a given sample that are isolated and detected using methods disclosed herein may be less than or equal to 100% 99.9%, 99%, 95%, 90%, 80%, 50%, or other appropriate percentage. Combinations of the forgoing are contemplated including, for example, fractions that are between or equal to 0.1% and 100%, or more preferably between 50% and 100%.

In some embodiments, at least one density media may be employed to permit density separation of the bound particle-target complexes. In some embodiments, a suitable density media may be a solution of iodixanol in water (e.g., Optiprep™ or Histodenz™) though other appropriate types of density media may be used. The density media may also include compounds such as cesium chloride, sodium metrizoate, sucrose, iodixanol, or any other suitable compound as the disclosure is not so limited. In some preferred embodiments disclosed herein, two or more density media may be employed to create an interface between layers at which bound particle-target complexes may be settled. In other embodiments, however, only one density media may be employed.

The layers of density media may be of any suitable height and/or volume as the disclosure is not so limited. In some embodiments, a suitable thickness of a layer of density media may be greater than or equal to 0.1 mm, 0.5 mm, 1 mm, 5 mm, 1 cm, 2 cm, 5 cm, 10 cm, 20 cm, 50 cm, 1 m, 5 m, or greater. The density media layer thickness may also be less than or equal to 10 m, 5 m, 1 m, 50 cm, 20 cm, 10 cm, or other appropriate thickness. Combinations of the forgoing are contemplated including for example, a density media thickness that is between or equal to 0.1 mm and 10 m, or more preferably between or equal to 1 cm and 10 cm, though other ranges are also possible. In some embodiments, a suitable volume of a layer of density media may be greater than or equal to 1 nL, 10 nL, 100 nL, 1 μL, 10 μL, 100 μL, 1 mL, 10 mL, 100 mL or greater. In some embodiments, a suitable volume of a layer of density media may be less than or equal to 1 L, 100 mL, 10 mL, 1 mL, or lesser. Combinations of the foregoing are contemplated including, for example, volumes between or equal to 1 nL and 1 L, or more preferably between 1 nL and 1 mL in some embodiments.

In some embodiments, it may be desirable to control the thickness of the interface between the layers of density media. A thicker interface may reduce additional aggregation of the targets and particles, which may be desirable in cases where binding of many particles to a target may cause its density to exceed the density of the media below the interface at which it is desired to isolate the target. For example, there may be a wait time during which the density media diffuse into each other to increase the thickness of the interface, or the density media may be mechanically mixed at the interface. In some embodiments, a suitable density media interface thickness may be greater than or equal to 0.1 mm, 0.5 mm, 1 mm, 2 mm, 5 mm, 1 cm, 2 cm, 5 cm, or greater. A suitable density media interface thickness may also be less than or equal to 10 cm, 5 cm, 2 cm, 1 cm, or lesser. Combinations of the foregoing are contemplated including, for example, thickness values between or equal to 0.1 mm and 10 cm, or more preferably between 0.1 cm and 1 cm in some embodiments.

In some embodiments, the density media may include additives to inhibit further aggregation of targets and particles. For example, blocking antibodies or antigens may be added to the density media to prevent further aggregation between targets and particles, without disrupting the complexes that are formed before they enter the density media.

The density of the density media may be selectively chosen to permit a density-based separation of the bound particle-target complexes relative to the background of the sample, the unbound targets, and/or the unbound particles. In some embodiments, a suitable density of a given density media may be greater than or equal to 0.95 g/ml, 1 g/ml, 1.05 g/ml, 1.1 g/ml, 1.15 g/ml, 1.2 g/ml, 1.25 g/ml, 1.3 g/ml, 1.35 g/ml, 1.4 g/ml. 1.45 g/ml, 1.5 g/ml, or greater. For example, in an embodiment where two density media are provided in a volume, the bottom density media may have a density of approximately 1.5 g/ml and the top density media may have a density of approximately 1.15 g/ml. In such an example, the bound particle-target complexes may have a density of 1.25 g/ml, thereby urging the bound complexes to settle at the interface formed by the two density media. While the above example is disclosed, the density media and bound particle-target complexes may be of any suitable density as the disclosure is not so limited. Moreover, the inventors have recognized that the bound complexes may have a spread in density as not every particle and/or target may be of the exact same density and/or size. The particles may also have a finite distribution of size and density, and may not be bound to the target in a 1:1 ratio. As such, different bound complexes may have varying sizes and/or density due to the polydispersity of the targets and particles and the ratio of particles to targets in the complexes. To ensure that such variations are accommodated for in the detection of targets, the inventors have recognized that a larger gap in density media density may be used to capture bound complexes with a finite distribution of effective density and size at the same interface. However, the inventors have also recognized that a larger density gap may result in the media becoming more viscous, which may impede the speed of separation and settling of the bound complexes.

It should be understood that while specific ranges of values for dimensional, volumetric, or other suitable parameters are provided herein, the disclosure is not limited to such examples, as values both greater and lesser than the various parameters disclosed herein are also contemplated.

In some embodiments, the density media may be loaded into a container or other device such that each density media is separated by a breakable, dissolvable, and/or removable barrier to prevent the density media from mixing with each other during storage, handling, or transportation. The one or more barriers may be broken mechanically, using a temperature change, under centrifugal force, or by other means. For example, the one or more barriers may consist of a fluid that is frozen under storage conditions and melts when the temperature is increased before use. In another example, the one or more barriers may consist of a plastic sheet that is drawn out through a tightly-fitting gap in the wall of the container or other device.

As disclosed herein, the embodiments of the proposed disclosure detail a variety of advantages over existing testing which may be inaccurate and unable to be completed in a timely fashion. The embodiments of the proposed disclosure may include benefits such as decreased cost relative to existing testing methods and integrated imaging analysis which may be incorporated into the embodiments disclosed herein. The inventors have also recognized that the embodiments of the invention may provide timely testing and results (e.g., isolation and examination of the targets in the sample approximately on the order of 1-3 hours). However, other potential and/or different benefits may also be provided.

In addition to the above, by contrast to traditional methods (e.g., blood culture), the proposed disclosure may be performed without culturing of the sample while still achieving rapid and reliable results. As a result of the accurate diagnostic results that may be achieved using the embodiments disclosed herein, patients may be able to be diagnosed in the earlier stages of various health conditions, which may reduce mortality rates among other benefits. In addition, several pathogens may be difficult to culture using traditional methods such as blood culture. For example, certain microbes may be slow growing under culture or require specific nutrients to grow. As the proposed disclosure permits detection of targets in a sample without culturing in some embodiments, previously hard-to-find microbes or other targets can be isolated and examined. The method and device of the proposed disclosure may also offer a lower limit of detection in the single digits of targets per milliliter. By comparison, blood culture is very slow for low concentration of targets (e.g., bacteria), and in some cases cannot be successfully used at low volumes (e.g., ˜0.5 ml) and low concentrations (e.g., ˜4 CFU/ml). By tuning the particles and incubation time, the embodiments disclosed herein provide the benefit of ensuring adequate targets may be captured regardless of the concentration of targets in the sample.

In some embodiments, the inventors have recognized that the proposed systems and methods may be used to supplement existing blood culture sampling. Specifically, when blood is drawn from a patient, a small amount (˜1 ml) can be added to the sample container and transferred to a benchtop device according to embodiments disclosed herein for incubation, separation, concentration, and analysis. As blood culture typically requires a patient blood draw of 20 ml, the proposed systems and methods may not use significant additional blood draw from the patient. The proposed disclosure may provide faster results than the blood culture, but may also be performed simultaneous to the blood culture. This may permit accurate analysis of blood results and early detection to be obtained for the patient. In addition, the inventors have recognized that certain vulnerable populations may benefit from reduced blood draw. For example, for populations of neonates and/or the elderly, the prospect of reduced blood draw while yielding accurate and timely results may prove extremely beneficial for these populations. Alternatively, larger amounts of blood (e.g., 5 ml, 10 ml, 15 ml, 20 ml, or 30 ml) may also be used.

In addition, one of the main drawbacks of blood culture as it is currently used is that it is sensitive to previous antibiotic administration, which slows down the reproduction of the bacteria. Since the embodiments disclosed herein do not rely on the reproduction of the bacteria, it can be used to establish the presence of bacteria in the bloodstream before, during, and after antibiotic administration. It would be a powerful tool to have feedback in order to cease antibiotic administration once the bacteria have been annihilated instead of waiting for a standardized dose to be administered. As drug-resistant bacteria become a greater concern for hospitals, being able to cease unnecessary antibiotic use is a major advantage of this invention.

As disclosed herein, the embodiments of the proposed disclosure may also be used in a variety of suitable applications. In some embodiments, a suitable application includes, but is not limited to food safety, sepsis, cell isolation, infectious diseases, water testing, rare cell detection, environmental monitoring, or any other suitable application as the disclosure is not so limited.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

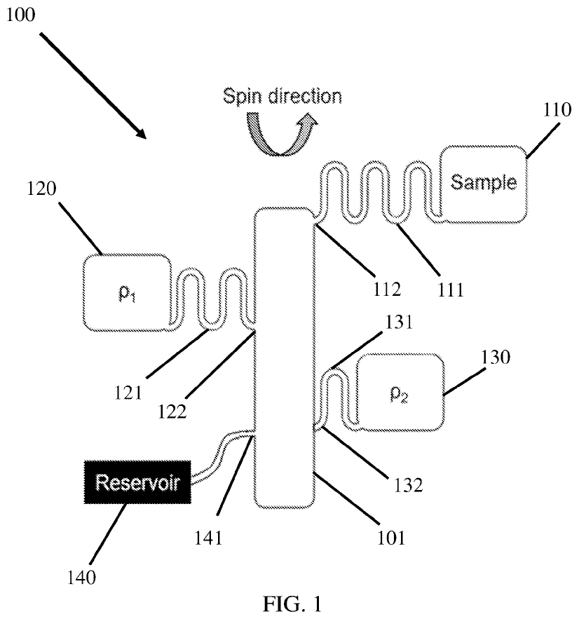

FIG. 1 presents an embodiment of a device 100, which may be a millifluidic device, used for loading of density media and a sample comprising targets. The device 100 includes a sample reservoir 110 comprising a sample, a first density media reservoir 120 comprising a first density media, and a second density media reservoir 130 comprising a second density media. In the embodiment of FIG. 1, the first and second density media may be of different densities. Each of these reservoirs (110, 120, 130) is in fluidic connection with a volume 101 which is configured to receive each of the reservoir's respective contents. The sample reservoir 110 may be connected to the volume 101 via channel 111, which may have a serpentine or other appropriate shape with a first length, and a sample fluidic inlet 112. The first density media reservoir 120 may be connected to the volume 101 via channel 121, which may have a serpentine or other appropriate shape with a second length, and a first density media fluidic inlet 122. The second density media reservoir 130 may be connected to the volume 101 via channel 131, which may have a serpentine or other appropriate shape with a third length, and a second density media fluidic inlet 132. As shown in FIG. 1, each of the serpentine channels (111, 121, 131) connecting the reservoirs (110, 120, 130) to the volume 101 are of different lengths. Namely, the second density media reservoir has the shortest length of the serpentine channels, followed by the first density media reservoir, and lastly the sample reservoir. As disclosed herein, the inventors have recognized that such a configuration may permit sequential loading of the respective reservoir contents in a selective fashion. In particular, in the embodiment of FIG. 1, the volume may be loaded in the following order: (1) the second density media, (2) the first density media, (3) the sample. Additionally, the associated inlets may be positioned at a location along a length of the volume such that the associate liquid (e.g., sample, density media, etc.) may be input into the volume within a portion where that liquid will be located during operation. Loading of the respective reservoir contents may be achieved by rotating and accelerating the volume 101 in a specific pattern to allow for the density media and sample to advance through the serpentine channels (111, 121, 131) and into the volume 101. While the above loading configuration is disclosed, the volume may be configured to be loaded in any suitable fashion as the disclosure is not so limited.

In the embodiment of FIG. 1, a plurality of particles may also be included in the volume (not shown). The plurality of particles may be loaded into the volume before, during, or following the loading of the density media and/or the sample. At least a portion of the particles may be configured to bind with at least a portion of the targets in the sample to form bound particle-target complexes. Upon formation of the bound complexes, the bound complexes may experience a density shift relative to the unbound targets and/or particles as well as the density media. For example, the bound complexes may settle at an interface formed at the location where the first and second density media interface one another. The unbound targets and/or unbound targets may then settle at the top and/or bottom of the density volume, or any other suitable location. The bound particle-target complexes may also be separated from the unbound targets and/or particles through application of a body force (e.g., centrifugal force). The volume may be centrifuged to permit separation of the bound complexes to a desired location (e.g. the interface of the density media). In such an example, the bound complexes may then be magnetically concentrated at an outlet 141 of the volume 101 where the bound complexes may then be extracted to an external reservoir 140 that is in fluid communication with the internal volume of the device. In some embodiments, this extraction may be controlled using suction, valves, magnetic forces, and/or any other appropriate extraction method and/or control. Alternatively, a separate extraction system, such as a pipette tube, extraction tube, or other extraction method may be used to extract the complexes from the interface location Upon extraction, the bound complexes may be analyzed using a variety of suitable methods discussed in greater detail below. While the embodiment of FIG. 1 shows the bound complexes being extracted to the external reservoir 140, the bound complexes may instead be retained within the volume 101 and analyzed through one or more viewing windows provided on the volume 101 (not shown).

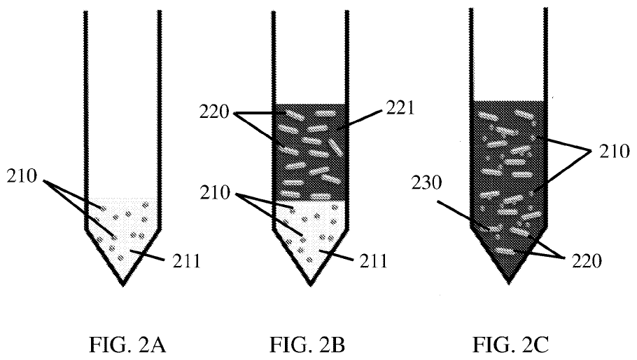

FIGS. 2A-2C present embodiments of a method of adding particles and targets to a container including a volume in which the desired separation may be performed and allowing the targets of the sample to incubate and form bound particle-target complexes in the sample container. In particular, FIG. 2A shows the step of adding a solution 211 containing a plurality of particles 210 into the bottom of the sample container. As disclosed herein, the particles 210 may include microspheres, which is the case in the embodiments of FIGS. 2A-C. FIG. 2B shows the next step of adding a sample 221 containing a plurality of targets 220 into the sample tube already containing the solution 211 of particles 210. FIG. 2C shows the next step of allowing the solution 211 of particles 210 and the sample 221 containing targets 220 to incubate and form a plurality of bound particle-target complexes 230.

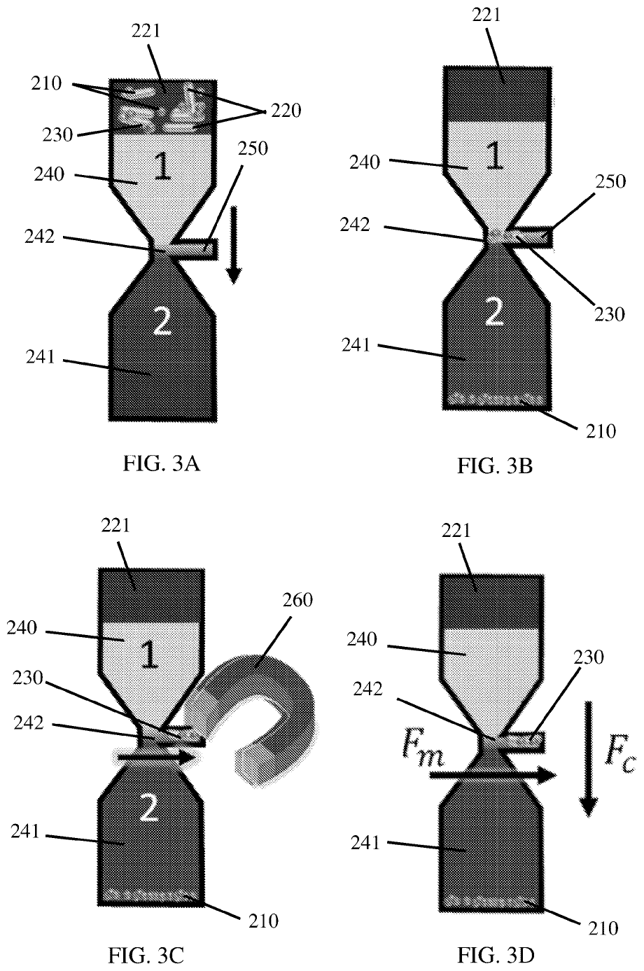

FIGS. 3A-3D present embodiments of a method of separating bound particle-target complexes from unbound targets and/or particles, and then concentrating the bound particle-target complexes at an outlet for extraction and analysis. The embodiments of FIGS. 3A-3D may be applied subsequent to the embodiments of FIGS. 2A-2C described above, though embodiments in which a separate incubation step is not performed are also contemplated. In FIG. 3A, a sample 221, which may be obtained from the steps of FIGS. 2A-2C, may be added into an internal volume of a device containing a first density media 240 and a second density media 241 which form an interface 242 therebetween. In FIG. 3B, the sample 221 is then centrifuged, or another body force may be applied, such that the bound particle-target complexes 230 settle at the formed interface 242 while the unbound particles 210 settle at the bottom of the volume and the unbound targets remain at the top of the volume in the incubated sample 221. In the next step of FIG. 3C, the bound particle-target complexes 230 may optionally be magnetically concentrated (via magnetophoresis) at a pre-positioned outlet 250 that is positioned at an expected location of the interface 242 within the internal volume of the device to permit extraction of the bound complexes 230. FIG. 3D depicts a force diagram of the steps of FIGS. 3B and 3C, where the centrifugal force, F_{C}, is shown to provide separation of the bound complexes 230 and the unbound targets 210 further down into the volume and the magnetic concentration force, F_{M}, is shown to provide concentration of the bound complexes 230 across the interface 242 towards the outlet 250. Moreover, the embodiments of FIGS. 3A-3D show the volume to have a tapering middle portion where the interface 242 is formed such that this middle portion of the device may have a maximum transverse dimension (e.g., a width or diameter) that is less than a maximum transverse dimension of adjacent portions of the internal volume of the device. The inventors have recognized that such a configuration may serve to reduce diffusion distances and increase magnetic forces applied to the bound particle-target complexes 230 across the interface 242, thereby making it easier to magnetically concentrate the bound complexes 230 at the outlet 250.

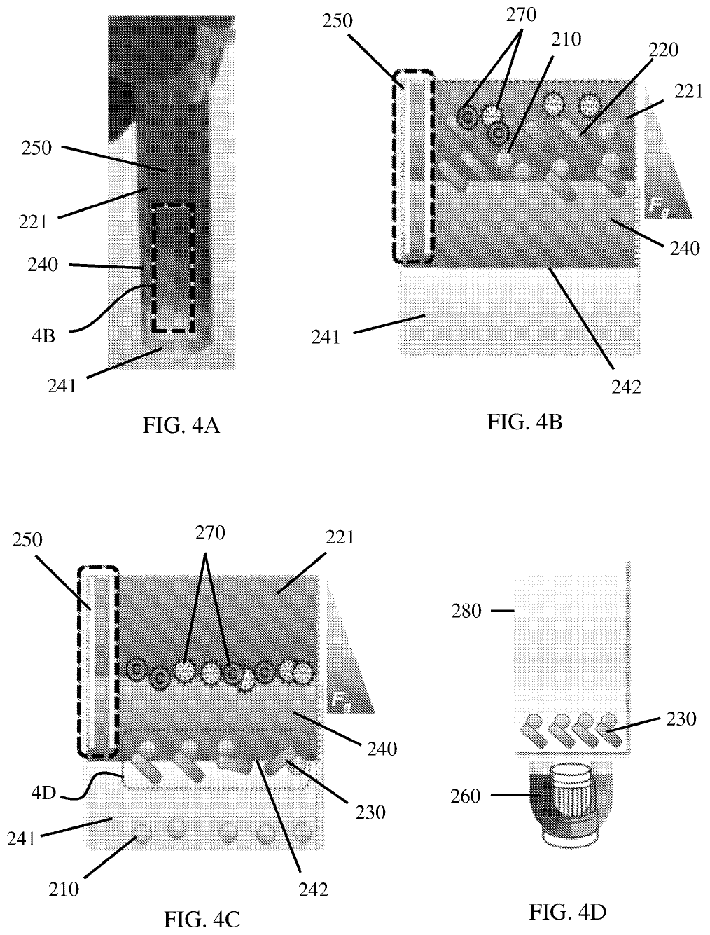

FIG. 4A depicts one embodiment image of a container (e.g., a tube) with a sample 221, a first density media 240, and a second density media 241 disposed in an interior volume of the container. The container also includes a pre-positioned outlet 250 disposed at an expected location of an interface between the first and second density media. FIG. 4B shows an enlarged view of the region 4B of FIG. 4A following an optional incubation step. In FIG. 4B, the sample 221 includes a plurality of particles in the form of magnetic beads 210 and a plurality of targets in the form of pathogens 220. The sample 221 also includes a plurality of non-target particles 270. As disclosed herein, the particles may be configured to bind with targets to form bound particle-target complexes. In FIG. 4C, which shows the embodiment of FIG. 4B following a separation step, a plurality of bound particle-target complexes 230 are formed. As a result of separation occurring in the tube, the bound complexes 230 experience a density shift relative to the unbound particles 210 and the non-target particles 270 such that the bound complexes 230 settle at the interface 242 while the unbound particles 210 settle at the bottom of the tube and the non-target particles 270 remain at the top of the tube. FIG. 4D is an enlarged view of the solution approximately located in region 4D of FIG. 4C, following extraction of the solution into an external container and a magnetic concentration step. In particular, FIG. 4D shows that the bound particle-target complexes 230 which settled at the interface 242 may be extracted to an external container 280 via the pre-positioned outlet 250. The bound particle-target complexes 230 may then be magnetically concentrated using a magnetic concentrator 260 to deposit the bound particle-target complexes 230 at the bottom of the external container 280. The bound particle-target complexes 230 may be then analyzed using a variety of suitable methods disclosed herein. The inventors have recognized that in some embodiments, it may be desirable to portion the respective volumes of the density media (240, 241) and the sample (221) such that the interface 242 is at or near the location of the pre-positioned outlet 250 to permit ease of extraction of the bound complexes 230. In addition or alternatively, the inventors have recognized that the length of the outlet 250 may be selectively chosen to ensure the interface 242 is positioned near the outlet 250. In either case, ensuring that the outlet 250 and the interface 242 are proximate one another during separation may provide benefits with easing extraction of the bound particle-target complexes 230 for analysis. As disclosed herein, instead of being extracted via the outlet 250, in some embodiments, the tube or other volume may include viewing windows for analysis of the concentrated bound complexes 230 while remaining in the volume or alternative extraction methods (e.g., automated or manual pipetting) may be used.

Once the bound particle-target complexes are isolated from the remainder of a sample using the embodiments disclosed herein, they may be analyzed using a variety of suitable methods. In some embodiments, the bound complexes may be analyzed via optical imaging using one or more viewing windows provided in the volume where the bound complexes are contained. For example, the bound particle-target complexes may be formed and settled at an interface between two density media, and the viewing windows may be provided in the side, top, and/or bottom of the volume for imaging analysis. The particles themselves may have certain optical properties that permit detection of the targets when bound. In some such embodiments, the targets may be detected via light scattering methods such as Raman spectroscopy. The particles may also have fluorescence or plasmonic properties to permit optical detection of the targets.

While methods of optical detection for analysis of the targets are disclosed above, non-optical detection methods may also be employed. In some embodiments, the particles bound to the targets may have magnetic or electric properties and the targets may be detected under a resulting magnetic or electric field, respectively. In some such embodiments, the particles may be actively manipulated such that movement of the particles bound to the targets may be detected within a certain field of view (e.g., via the one or more viewing windows which may be provided in the volume). In view of the above, the particles may have a variety of suitable properties to permit detection of the targets to which the particles are bound.

The bound particle-target complexes may also be extracted and analyzed as disclosed above. In some such embodiments, a variety of downstream analysis methods may be employed to analysis the targets in the sample. In some embodiments, the targets may be analyzed using assays (e.g., polymerase chain reaction (PCR) or other appropriate assay), flow cytometry, mass spectrometry, microscopy, and/or cell culture with single-cell visualization.

The following examples are intended to illustrate certain embodiments of the present disclosure, but do not exemplify the full scope of the disclosure.

### EXAMPLES

In one example, bacteria were isolated from a background of whole blood by tagging them with antibody-functionalized magnetic beads and using both the density shift and magnetic affinity of the tagged bacteria to separate them from blood cells and excess magnetic beads. Magnetic beads are added to a spiked whole blood sample and incubated to permit binding. The tagged bacteria are separated from both mammalian cells and unbound magnetic beads by use of a volume with density media and centrifugation. The bound bacteria are localized at a distinct interface and can be extracted, concentrated magnetically, and examined using fluorescence microscopy to confirm the presence of bacteria in the blood.

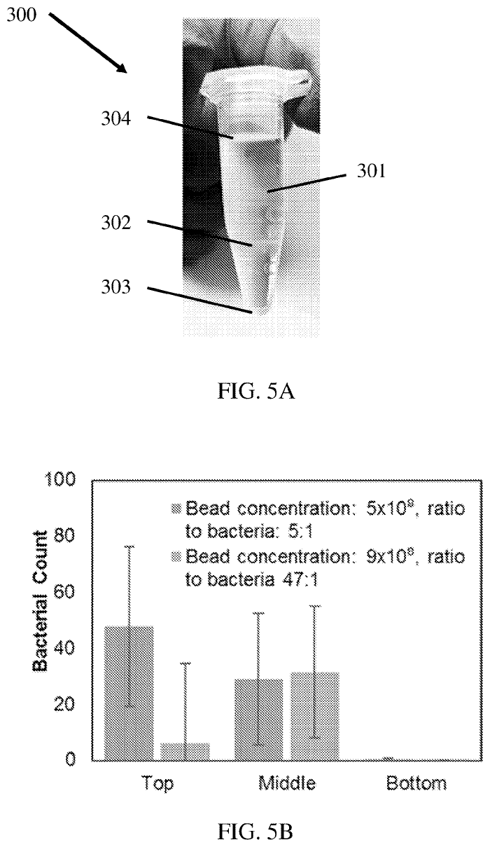

GFP-transfected E. coli were incubated with streptavidin-coated magnetic microspheres procured from CD Bioparticles (WHM-S101, with 0.4-0.6 μm diameter, 1.58 g/mL density) coated with biotin-conjugated anti-E. coli polyclonal antibodies (Abcam, ab68451) or BSA-labeled control beads for two hours at room temperature, with gentle rotation to promote bacteria-bead interactions through the natural gravity-induced settling of beads. The bound bacteria from the denser background of unbound beads and lighter unbound bacteria by layering the mixture in a volume including Optiprep density media diluted to 1.13 g/mL and 1.28 g/mL and centrifuged it at 3000 RCF for 10 minutes. A fraction of the bacteria were localized at the middle interface, where only bacteria-bead pairs were observed; unbound magnetic beads settled at the bottom of the volume. FIGS. 5A and 5B shows the results of the centrifugal separation step, with bacteria only at the top and middle interfaces. After the density gradient centrifugation, the middle interface was extracted and the bound bacteria were concentrated.

Special extraction tubes were prepared by gluing tubing with an inner diameter of approximately 1 mm to the inside of 1.5 ml centrifugation tubes. The tubing was placed 1.15 cm from the bottom of the centrifugation tube in order to enable the extraction at the interface of the density media. The volume was prepared using 250 μL of 60% Optiprep and 350 μL of 25% Optiprep. Then the 500 μL sample was gently added to the top of the volume and the tubes were centrifuged at 3000K RCF for 25 minutes to separate the particles: unbound beads to the bottom of the volume, bound bacteria at the interface, and blood above the first Optiprep layer. After centrifugation, a 20-gauge blunt-tipped needle and a 1 ml syringe were used to extract approximately 285 μL (200 μL plus the volume of the needle) from the 25%/60% Optiprep interface via the tubing.

This extract was placed in a 1.5 mL conical tube over an array of magnets and allowed to settle for 1.5 hours. Then, approximately 265 μL of the supernatant was removed, leaving 20 μL of concentrated bound bacteria behind. This volume of concentrated bound bacteria was mixed using a pipette and then 3 μL was pipetted onto a glass slide, covered with a coverslip, and examined under fluorescein isothiocyanate (FITC) fluorescence microscopy to count the number of bacteria retrieved.

While large variations were observed over these initial samples, the tests illustrated that bacteria can be made effectively denser by binding with antibody-labeled magnetic beads, separated from unbound magnetic beads, and then concentrated to a measurable level.

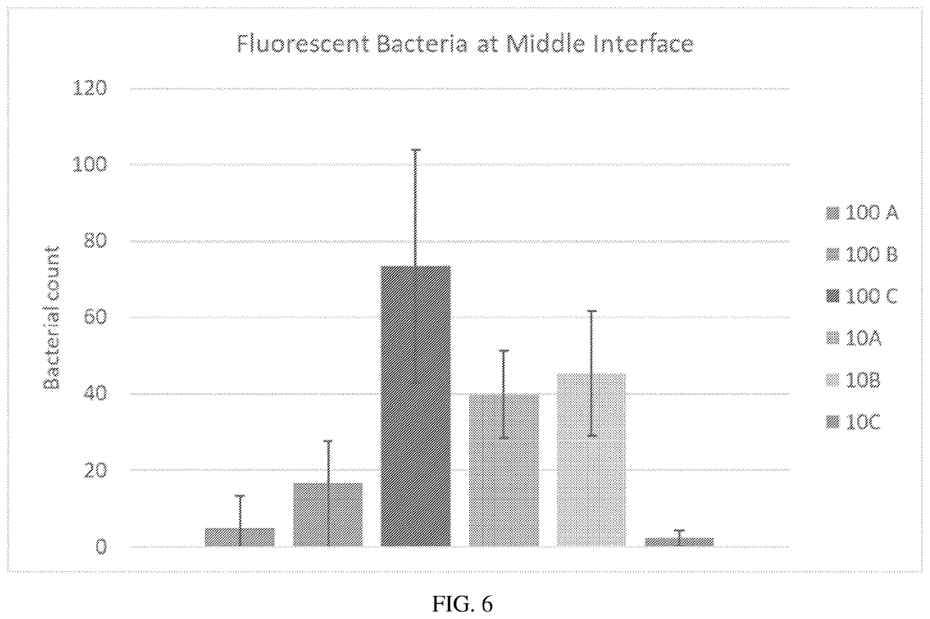

FIG. 6 shows results for an experiment where bacterial counts at the middle interface of density media were recorded using three different samples with a concentration of 108 bacteria in a buffer. The buffer was mixed with beads to either a 10:1 or 100:1 bead to bacteria ratio for each of the three samples. The captured bacteria were observed at up to 70 bacteria per field of view, as shown in FIG. 6.

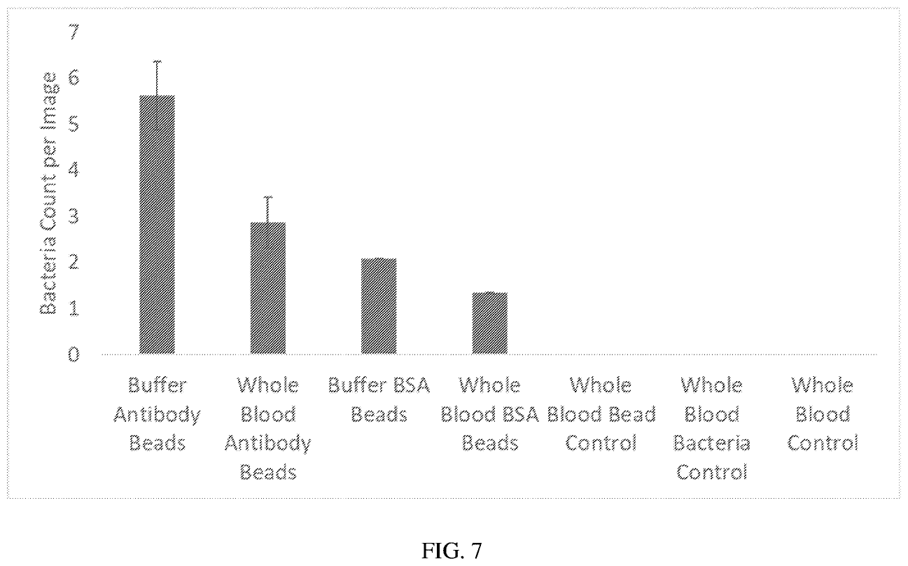

Separation and concentration in whole blood was achieved using the same processes. E. coli was spiked into whole blood and then the incubation, separation, and concentration steps were performed according to embodiments described herein. As seen in FIG. 7, the controls in this experiment confirm that bacteria were only found at the interface if they were bound to magnetic beads.

The primary test design used E. coli as the target cells and a polyclonal anti-E. coli antibody from Abcam (AB68451) to functionalize magnetic beads from CD Bioparticles (0.4-0.69 μm, 1.58 g/ml density, streptavidin-coated). E. coli cells are generally cylindrical cells with dimensions 0.5 μm wide by 1-2 μm long and density of approximately 1.1 g/ml. Other cells in blood range from 6-10 μm with densities from 1.06 to 1.09 g/ml. Using this set-up, it was demonstrated that bead bound to approximately 20% of the bacteria before optimization. Bead-bacteria binding has also been verified using flow cytometry

The effectiveness of the disclosed technique may depend on the kinetics of binding. The concentrations of target cells, background cells, and microspheres impact the timing and capture efficiency of the device. Mixing reduces the time to bind.

Once the microspheres have bound to the target cells, the bound complexes may be separated from background cells and unbound beads. The key principle is that only the target cells/particle pairs or clusters experience a zero-sum body force at an interface or location, which causes localization of only target cells bound to particles at that interface. One way that this can be achieved is through density separation using discrete density media. Given that the primary embodiment uses high density magnetic beads (ρ>1.45 g/ml), the effective density shift is exploited to differentiate the bacteria from non-target cells and unbound beads. However, other properties of the microsphere-target cell complexes could be used to separate, such as size, shape, charge (electrophoresis), polarizability (dielectrophoresis), or magnetic properties could be used to achieve this separation as well. The desired localization could be achieved through a combination of two or more of forces that depend on properties such as those listed above.

To employ the primary embodiment's technique of perturbed density interfacial capture (PDIC) to separate the target cells from the background, a discrete density gradient is formed such that the effective density of bound target cells is greater than the less dense media and less that the densest media. Careful selection of the microspheres and density media can result in unbound microspheres at the very bottom of the volume of density media, microsphere-target cell complexes at the middle (capture) interface, and non-target cells at the top interface.

For the primary use case of bacteria in a blood sample with antibody-functionalized magnetic beads, separation may occur most quickly in cases where the differences in density between the following particles or media are maximized. Some examples of changes in target (e.g., bacteria) density resulting from binding to magnetic beads are as follows. Unbound beads and erythrocytes (density=1.12 g/ml). This defines the overall density range available for the process, as unbound beads fall at the bottom of the density column while erythrocytes are the densest blood component and may desirably remain at the top of the volume. One bacteria bound to a single bead and erythrocytes: A bacteria bound to a single bead must be sufficiently denser than an erythrocyte to fall through the less dense media to the capture interface, and the larger the difference, the quicker the separation will occur. One bacterium completely coated in beads and unbound beads: It is desirable that all bacteria remain at the capture interface and do not descend through the denser media. The larger the difference in density of these two particles, the more rapidly the unbound bead background may fall through the denser media leaving a clear target population at the capture interface.

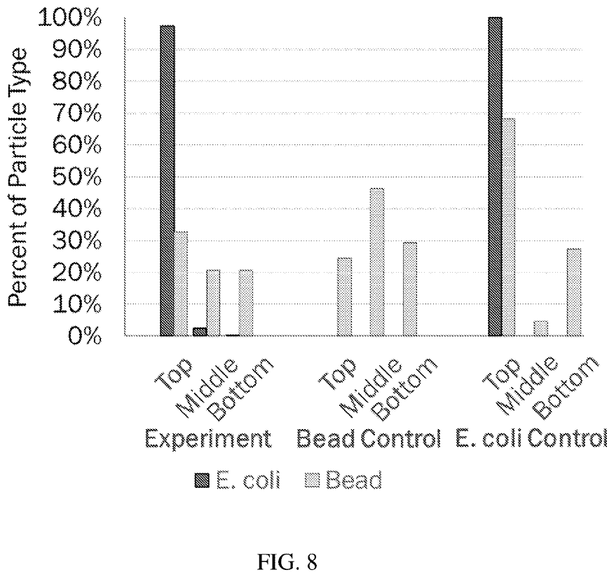

PDIC was successfully demonstrated using the macroscale prototype. FIG. 8 shows the presence of E. coli at the middle (capture) interface in only the experimental treatment, which suggests that beads were successfully bound to bacteria and dragged them to the capture interface by altering their effective density. The single E. coli at the bottom interface was likely lysed. For the macroscale prototype, for density media heights of 7 mm and 5 mm, separation can be achieved in under ten minutes. The equation that governs the separation time is:

$${t}_{separation} = \sum \frac{18 {h}_{i} {η}_{i}}{{d}^{2} ( {ρ}_{particle} - {ρ}_{i} ) g}$$

Where i indicates the layers of media: for each layer, there is a height (h), density (ρ), and viscosity (η). The gravitational force created by the centrifuge is g, d is the diameter of the particle, and ρ_{particle} indicates the density of bound complexes or unbound microspheres. The time is derived from the velocity achieved by a particle subjected to both buoyancy forces and Stokes drag.

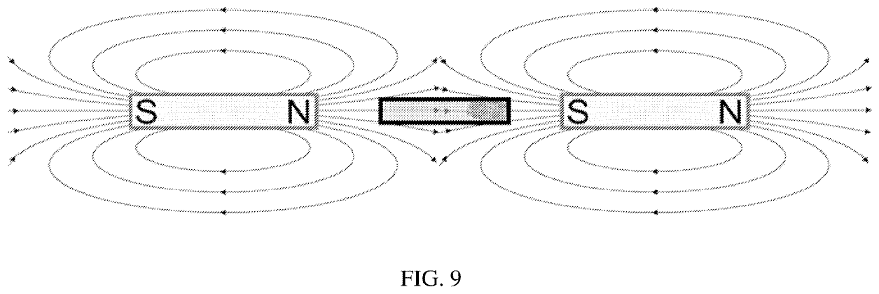

After the target cells were separated from the background and confined to a smaller volume, they were further concentrated before detection or analysis. Given that the intended sample volume may be about 1 ml, it is possible that there are only 10 CFUs that would be caught at the density interface. Therefore, in some instance, a magnetic field may be used to concentrate the bound bacteria, or other targets into a smaller volume. However, the concentration step could be accomplished through droplet sorting, flow focusing, or other methods. An illustration of a proposed magnetic setup is shown in FIG. 9 using permanent magnets. A maximum separation time of the bound bacteria across a 15 mm tube length was calculated to be 3370 seconds, or approximately 56 minutes. 15 mm is much larger than the anticipated distance, so it is clear that this separation can be done in under an hour.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method of detecting targets in a sample, the method comprising:
binding at least a portion of a plurality of particles to at least a portion of a plurality of targets within a volume such that a plurality of bound particle-target complexes are formed, wherein a density of the bound particle-target complexes is different than both unbound targets and unbound particles; and
separating the bound particle-target complexes from both the unbound targets and the unbound particles using at least one density media.

2. The method of claim 1, wherein the density of the bound particle-target complexes is greater than the density of the unbound targets and less than a density of the unbound particles.

3. The method of claim 1, wherein the density of the bound particle-target complexes is greater than a density of the unbound particles and less than a density of the unbound targets.

4. The method of claim 1, further comprising applying a body force to separate the bound particle-target complexes from the unbound targets and the unbound particles.

5. The method of claim 1, further comprising magnetically concentrating the bound particle-target complexes at an outlet and extracting the bound particle-target complexes from the outlet.

6. The method of claim 1, wherein the at least one density media comprises a first density media and a second density media which form an interface between the first and second density media, wherein the density of the particle-target complexes is greater than the first density media and less than the second density media, and further comprising settling the bound particle-target complexes at the at least one interface.

7. The method of claim 6, further comprising extracting the bound particle-target complexes from the interface.

8. The method of claim 1, further comprising analyzing the bound particle-target complexes through one or more viewing windows associated with the volume.

9. The method of claim 1, wherein the targets include at least one selected from the group of cells, viruses, bacteria, plankton, food pathogens, fungi, protozoa, exosomes, cell organelles, microplastics, waste particulates, metal particles, and soil particulates.

10. The method of claim 8, wherein the cells include at least one selected from the group of blood cells, cancer cells, immune cells, and plant cells.

11. The method of claim 1, wherein the particles are at least one selected from the group of air bubbles, liquid droplets, magnetic particles, core shell particles, metal particles, ceramic particles, plasmonic coated particles, multiphase particles, anti-fouling coated particles, polymer brush coated particles, and gels.

12. The method of claim 1, wherein binding at least the portion of the plurality of particles to at least the portion of a plurality of targets includes binding using at least one selected from electrostatic binding, magnetic binding, capillary force binding, surface adhesion binding, chemical binding, and aptamer binding.

13. A device for detecting targets in a sample comprising:
a volume;
at least one density media reservoir configured to contain at least one density media, wherein the at least one density media reservoir is in fluid communication with the volume;
a sample reservoir configured to contain a sample including a plurality of targets, wherein the sample reservoir is in fluid communication with the volume; and
a plurality of particles disposed in the volume, wherein the plurality of particles are configured to bind to the plurality of targets to form bound particle-target complexes, wherein a density of the bound particle-target complexes is different density than both unbound targets and unbound particles.

14. The device of claim 13, wherein the bound particle-target complexes are configured to be separated from both the unbound targets and the unbound particles by application of a body force.

15. The device of claim 13, wherein the at least one density media comprises a first and a second density media which form an interface between the first and second density media, wherein the density of the bound particle-target complexes is greater than a density of the first density media and less than a density of the second density media.

16. The device of claim 15, further comprising an outlet in fluid communication with the volume, wherein the outlet is configured to extract the bound particle-target complexes from the interface.

17. The device of claim 16, further comprising a magnetic concentrator, wherein the magnetic concentrator is configured to concentrate the bound particle-target complexes at the outlet.

18. The device of claim 13, further comprising one or more windows associated with volume.

19. The device of claim 13, wherein the targets are at least one selected from the group of cells, viruses, bacteria, plankton, food pathogens, fungi, protozoa, exosomes, cell organelles, microplastics, waste particulates, metals, and soil particulates.

20. The device of claim 19, wherein the cells include at least one selected from the group of blood cells, cancer cells, immune cells, and plant cells.

21. The device of claim 13, wherein the particles are at least one selected from the group of air bubbles, liquid droplets, magnetic particles, core shell particles, metal particles, ceramic particles, plasmonic coated particles, multiphase particles, anti-fouling coated particles, polymer brush coated particles, and gels.

22. The device of claim 13, wherein the plurality of particles are configured to bind to the plurality of targets using at least one selected from electrostatic binding, magnetic binding, capillary force binding, surface adhesion binding, chemical binding, and aptamer binding.

23. The device of claim 13, wherein an average maximum transverse dimension of the plurality of particles is between or equal to 5 nm and 1,000 μm.
